(19) United States
(12) Patent Application Publication
(10) Pub. No.: US 2024/0225654 A9
(43) Pub. Date: Jul. 11, 2024

(54) THERAPEUTIC TREATMENT DEVICE WITH BRAIDED MICROVALVE OCCLUDER HAVING COLLAPSIBLE COVER

(71) Applicant: TriSalus Life Sciences, Inc., Westminster, CO (US)

(72) Inventors: Michael Brick Markham, Boulder, CO (US); David Benjamin Jaroch, Arvada, CO (US)

(73) Assignee: TriSalus Life Sciences, Inc., Westminster, CO (US)

(21) Appl. No.: 17/970,329

(22) Filed: Oct. 20, 2022

(51) Int. Cl. A61B 17/12; A61M 25/00

(52) U.S. Cl. CPC A61B 17/1204; A61B 17/12109; A61B 17/12172; A61M 25/0075; A61B 2017/1205; A61M 2025/0002

(57) ABSTRACT

A treatment device for use in a vessel includes a catheter and a microvalve. A flexible low friction reconfigurable cover is attached at or adjacent a distal end of the microvalve. The cover is deployed between the occluder and the vessel wall to intervene between the occluder and the vessel wall during movement of the occluder relative to the vessel wall to reduce occurrence of vascular spasm.

### CROSS-REFERENCE TO RELATED PATENTS

This application is related to co-owned U.S. Pat. Nos. 8,696,698 and 10,588,636, which are hereby incorporated by reference herein in their entireties.

### BACKGROUND OF THE INVENTION

### 1. Field of Invention

The disclosure relates generally to catheters for delivering a therapeutic treatment into a blood vessel. More particularly, this disclosure relates to catheters having microvalves at the distal end thereof to increase penetration of the therapeutic treatment into targeted blood vessels and reduces reflux of the therapeutic treatment into non-targeted blood vessels.

### 2. State of the Art

Intravascular therapeutic treatments are often clinically delivered to treat a wide range of diseases. By way of example, intravascular embolization, chemo-embolization, and radio-embolization therapies are used to treat a range of diseases, such as hypervascular liver tumors, uterine fibroids, secondary cancer metastasis in the liver, pre-operative treatment of hypervascular menangiomas in the brain and bronchial artery embolization for hemoptysis.

Non-targeted delivery of various therapies can lead to adverse events and morbidity. In addition, non-targeted delivery suggests that the intended target of the delivery is not receiving the full dose of therapy.

Infusion with a standard infusion microcatheter allows bi-directional flow. That is, the use of a microcatheter to infuse a therapeutic agent allows blood and the infused therapeutic agent to move forward in addition to allowing blood and the therapeutic agent to be pushed backward (reflux). Reflux of a therapeutic agent causes non-target damage to surrounding healthy organs. In interventional oncology therapeutic procedures, the goal is to bombard a cancer tumor with either radiation or chemotherapy. It is important to maintain forward flow throughout the entire vascular tree in the target organ in order to deliver therapies into the distal vasculature, where the therapy can be most effective. This issue is amplified in hypovascular tumors or in patients who have undergone chemotherapy, where slow flow limits the dose of therapeutic agent delivered and reflux of agents to non-target tissue can happen well before the physician has delivered the desired dose.

The pressure in a vessel at multiple locations in the vascular tree changes during a therapeutic infusion procedure. Initially, the pressure is high proximally, and decreases over the length of the vessel. Forward flow of therapy occurs when there is a pressure drop. If there is no pressure drop over a length of vessel, therapy does not flow downstream. If there is a higher pressure at one location, such as at the orifice of a catheter, the therapeutic therapy flows in a direction toward lower pressure. If the pressure generated at the orifice of an infusion catheter is larger than the pressure in the vessel proximal to the catheter orifice, some portion of the infused therapeutic therapy travels up stream (reflux) into non-target vessels and non-target organs. This phenomenon can happen even in vessels with strong forward flow if the infusion pressure (pressure at the orifice of the catheter) is sufficiently high.

In clinical practice with a standard infusion catheter, the physician attempts to infuse the therapeutic agent with pressure that does not cause reflux. In doing this, the physician slows the infusion rate (and infusion pressure) or stops the infusion completely. The clinical impact of current infusion catheters and techniques is two fold: low doses of the therapeutic agent is delivered and there is poor distal penetration into the target vessels.

Additionally, reflux can be a time-sensitive phenomenon. Sometimes, reflux occurs as a response to an injection of the therapeutic agent, where the reflux occurs rapidly (e.g., in the time-scale of milliseconds) in a manner which is too fast for a human operator to respond. Also, reflux can happen momentarily, followed by a temporary resumption of forward flow in the blood vessel, only to be followed by additional reflux.

Various devices have been proposed to increase distal penetration while preventing reflux. For example, co-owned U.S. Pat. No. 8,696,698, which has been incorporated by reference herein, describes a microvalve infusion system for infusing a therapeutic agent that has a dynamically adjustably filter valve coupled at a distal end of a delivery catheter. The delivery catheter and filter valve self-expand when deployed from a delivery catheter. The filter valve is naturally spring biased by its construction of filamentary elements to automatically partially expand within a vessel when it is deployed from the outer catheter, and is coated with a porous polymer coating that has a pore size sufficiently small to filter a therapeutic agent. In view of the construction, upon infusion, an increase in fluid pressure results within the filter valve and causes the filter valve to open, extend across a vessel, and thereby prevent reflux of the infused therapeutic agent. In addition, as the fluid is pressurized through the delivery catheter and into the filter valve, the downstream pressure in the vessel is increased which facilitates maximum uptake into the target tissue for therapeutically delivered agents. Further, the filter valve is responsive to local pressure about the valve which thereby enables substantially unrestricted forward flow of blood in the vessel, and reduces or stops reflux (regurgitation or backward flow) of therapeutic agents which are introduced into the blood.

However, devices in U.S. Pat. No. 8,696,698 have certain characteristics that may not always be advantageous for a given situation. The filter valve devices disclosed are generally well-adapted where tracking the occluder into small vessels in not a significant requirement; trackability in tortuous branching vasculature can be limited. The distal end of the device in a collapsed, undeployed state is defined by the size of the deployment catheter, which can be significantly larger than the catheter that supports the filter valve and significantly larger than the outer diameter of a guidewire used to the guide the microvalve to the target location within the vessel. As such, tracking the filter valve into the smaller vascular branches may not be optimal. In addition, once the device is tracked to a treatment location, deployment of the filter valve requires that the frictional force between the filter valve and the outer deployment be overcome.

Co-owned U.S. Pat. No. 10,588,636, previously incorporated herein, described a microvalve infusion system for infusing a therapeutic agent and which addresses device trackability. Referring to Prior Art FIG. 1, the system 10 includes a flexible infusion catheter 12 having a hub 14 at a proximal end 16 and a filter valve occluder 18 coupled to a distal end 20. The filter valve occluder 18 includes braided elastic strands 22 that each include a proximal portion 24, a central portion 26, and a distal portion 28. The proximal portions 24 are attached circumferentially about at an outer surface 30 of the catheter 12 at a location proximal of a distal orifice 32 of the catheter, the central portions 26 extend radially outward and toward the orifice 32, and the distal portions 28 of the strands are inverted back into the filter valve occluder 18 and coupled circumferentially about the outer surface 30 of the catheter 12. The proximal and central portions 24, 26 of the strands 22 are coated in a polymeric filter coating 34 that extends between and across the strands 18. The distal portions 28 of the strands 22 are uncoated in the polymeric filter. A variation on this microvalve infusion system 10 is commercially produced and sold as the TRINAV® infusion system by Trisalus Life Sciences, Inc., Westminster, CO. In use, the microvalve infusion system can be deployed from an introducer sleeve 36 at a target vessel location; but is not required to be used with an introducer sleeve. Rather, the infusion system can be advanced over a guidewire without any introducer sleeve, providing good results with trackability. Upon introduction in the vessel, the filter valve occluder 18 has been shown to dynamically operate in sync with the cardiac cycle and preserve more than 70 percent of antegrade blood flow in a vessel past the microvalve occluder, while providing intended retrograde blockade of therapeutics. Further, the design permits atraumatically increasing the pressure of therapeutic infusion into local resistive tumor vessels to enable deeper perfusive delivery of therapeutics.

It has been identified that when some microvalve occluders are longitudinally displaced within a vessel, particularly when the occluders are constructed at a larger size, there is significant resistance to advancing the microvalve occluder in the vessel. When the microvalve occluder is advanced, the microvalve occluder is deformed, causing an increased surface area to be in contact with the vessel wall. There is drag at the interface of the occluder surface and the vessel wall. Therefore, with more surface in contact with the vessel wall, more drag is generated.

Such drag can result in trauma or irritation in the arteries, which can cause vascular spasm. Vascular spasm is a condition in which the smooth muscle of the arteries contracts. This can occur locally or trigger a cascading effect along a length of the vessel. This constriction can drastically reduce blood flow in the vessel and subsequently the distal pressure in the vessel. If this occurs proximal to a microvalve occluder, a lack of antegrade flow will be available which can hinder the downstream flow post-infusion, creating an effect similar to an occlusive balloon. If the spasm is distal to the microvalve occluder, distal flow can be substantially reduced, thereby inhibiting delivery of the therapeutic agent. Additionally, tracking a microvalve occluder forward or backward through a spasm can lead to device damage or greater vascular injury.

### SUMMARY OF THE INVENTION

An infusion device is provided that includes a catheter and an occluder. The catheter has a proximal end, a distal end with a distal tip, and a lumen extending from the proximal end to the distal tip, and opening at a distal orifice through the distal tip. The occluder can be a microvalve.

In accord with an aspect of the infusion device, a flexible cover is attached at or adjacent the distal end of the microvalve. The flexible cover may be formed from a smooth, lubricous sheet material. The flexible cover is adapted to be interposed between the occluder and the patient's vessel wall to provide a low friction surface against the vessel wall when the occluder is advanced within the vessel. The cover reduces or prevents occurrence of vascular spasm during device tracking and navigation.

The cover is preferably deployed in response to resistance from forward motion as the occluder is advanced through a vessel. Then, when the motion is stopped, the force of the antegrade flow within the vessel moves the cover away from the occluder and the occluder functions as intended.

### BRIEF DESCRIPTION OF DRAWINGS

Prior art FIG. 1 is a side elevation of a prior art infusion system.

FIG. 2 is side elevation of an infusion system according to an embodiment described herein.

FIG. 3 is an enlarged view of the distal end of the infusion system shown in FIG. 2.

FIGS. 4 through 6 are schematic illustrations of operation of the infusion device in a vessel: while being advanced through the vessel (FIG. 4); while stationary and subject to antegrade fluid flow (FIG. 5); and during infusion of a therapeutic agent through the infusion device (FIG. 6).

FIGS. 7 through 9 are schematic illustrations of operation of a variation of the infusion device in a vessel: while being advanced through the vessel (FIG. 7); while stationary and subject to antegrade fluid flow (FIG. 8); and during infusion of a therapeutic agent through the infusion device (FIG. 9).

FIGS. 10 through 12 are schematic illustrations of operation of a variation of the infusion device in a vessel: while being advanced through the vessel (FIG. 10); while stationary and subject to antegrade fluid flow (FIG. 11); and during infusion of a therapeutic agent through the infusion device (FIG. 12).

### DETAILED DESCRIPTION OF THE PREFERRED EMBODIMENTS

With reference to the human body and components of the devices and systems described herein which are intended to be hand-operated by a user, the terms “proximal” and “distal” are defined in reference to the user's hand, with the term “proximal” being closer to the user's hand, and the term “distal” being further from the user's hand, unless alternate definitions are specifically provided.

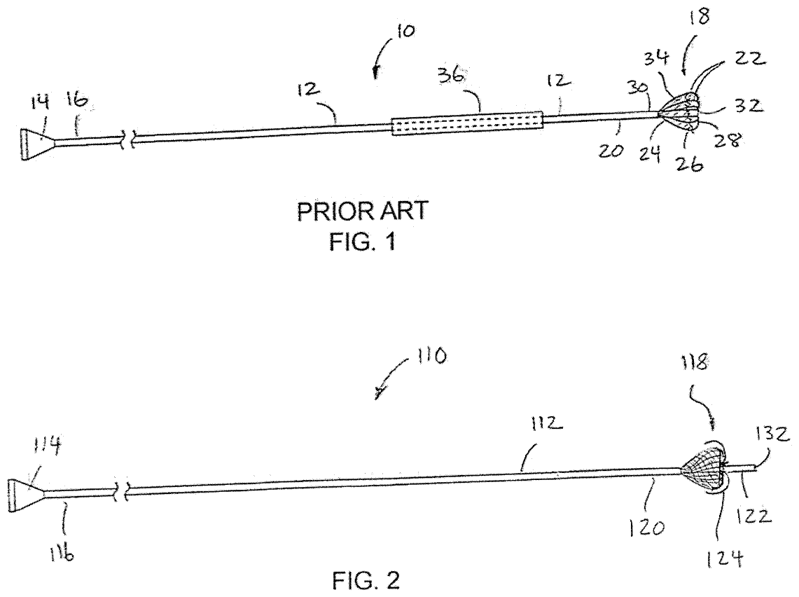

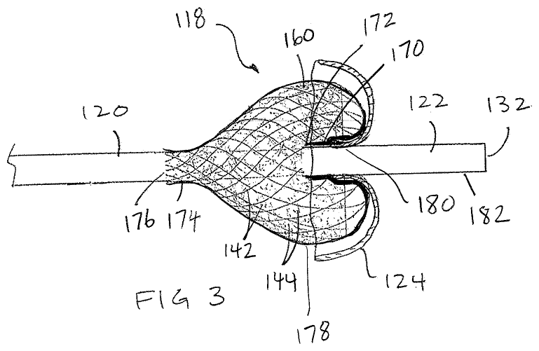

A first exemplary embodiment of an infusion device 110 according to the invention is shown in FIGS. 2 and 3. It is noted that respective portions of the system illustrated in such figures are not shown proportional to their intended size, but rather that the distal portion is illustrated significantly enlarged for purposes of explanation. As shown in FIG. 2, the infusion device 110 includes a flexible catheter 112 having a proximal end 116 and a distal end 120, a hub 114 coupled to the proximal end 116 of the catheter, and a vessel occluder 118, described in more detail below, coupled to the distal end 120 of the catheter 112. In accord with an aspect of the infusion system, a deployable cover 124, also described in more detail below, is provided for shielding the vessel wall of the patient from an outer surface of the vessel occluder 118 while the infusion device 110 is tracked over a guidewire and/or otherwise navigated to a target location within a vessel of the patient.

The catheter 112 is between two and eight feet long, and has an outer diameter of between 0.67 mm and 3 mm (corresponding to catheter sizes 2 French to 12 French), and is made from a liner made of fluorinated polymer such as polytetrafluoroethylene (PTFE) or fluorinated ethylene propylene (FEP), a braid made of metal such as stainless steel or nickel titanium alloy, or a polymer such as polyethylene terephthalate (PET) or liquid crystal polymer, and an outer coating made of a polyether block amide thermoplastic elastomeric resin such as Pebax®, polyurethane, polyamide, copolymers of polyamide, polyester, copolymers of polyester, fluorinated polymers, such as PTFE, FEP, polyimides, polycarbonate or any other suitable material, or any other standard or specialty material used in making catheters used in the bloodstream.

The hub 116 may include a leur connector or other standardized connector. An infusion lumen extends from the hub 116 to a distal tip 122 of the catheter and opens at a distal orifice 132 of the catheter so that the hub 116 is adapted for delivery of a therapeutic agent from outside the body of the patient to a target vessel (artery or vein) in the patient. The hub 116 is preferably also adapted to facilitate advancement of a guidewire through the infusion lumen and/or coupling of a syringe for infusion of the therapeutic agent through the infusion lumen. Any hub suitable for at least facilitating delivery of a therapeutic agent into the infusion lumen can be utilized.

The occluder 118 is preferably a dynamic microvalve made as follows. Referring to FIG. 3, a braid of multiple strands 142 in tubular form is created or provided. The tubular braid is comprised of multiple metal (e.g., stainless steel or nickel-titanium alloy) or polymer filaments or strands 142 in a tubular braided construction, which form a substantially closed shape when deployed and not subject to outside forces. Where polymeric filaments are utilized, the filaments 142 may be composed of PET, polyethylene-napthalate (PEN), liquid crystal polymer, fluorinated polymers, nylon, polyamide or any other suitable polymer. If desired, when polymeric filaments are utilized, one or more metal filaments may be utilized in conjunction with the polymeric filaments. According to one aspect of the invention, where a metal filament is utilized, it may be of radio-opaque material to facilitate tracking the filter valve occluder 118 and its configuration within the body. The filaments 142 are not bonded to each between their ends so to enable them to move relative to each other between their ends. The filaments are spring biased (i.e., they have “shape memory”) to assume a desired crossing angle relative to each other. Between each two adjacent sets of crossing filaments, diamond-shaped interstices 144 are formed, with the crossing of the filaments defining vertices of the diamond-shaped interstices. The specific shape and size of the diamond-shaped interstices is determined by the braid angle between crossing filaments in the tubular braid.

The diameter of the filaments 142 are chosen in the range of 0.025 mm to 0.127 mm, although other diameters may be utilized. Preferably, the pitch angle (i.e., the crossing angle assumed by the braided filaments in the fully open deployed position) is chosen in the range of 100° to 150°, although other pitch angles may be used.

In accord with one aspect of the infusion device, a primer is provided to the strands 142 of the tubular braid. The primer functions to facilitate adhesion of the polymer coating, discussed below, and to maintain stability and polymer on the filaments to avoid degradation over time. Exemplar primers include a Tecoflex® polyether polyurethane, polyamic acid, and parylene.

Then, a polymer coating 160 is applied over the elastic strands 142 and across the diamond-shaped interstices 144 formed between the strands. The polymer 160 can be coated onto the braid by any of several methods, including by spraying, spinning, electrospinning, bonding with an adhesive, thermally fusing, mechanically capturing the braid, melt bonding, dip-coating, or any other desired method, to form a coating suitable for use as a filter. The filter can either be a material with pores such as ePTFE, a solid material that has pores added such as polyurethane with laser drilled holes, or the filter can be a web of very thin filaments that are laid onto the braid.

Where the polymer filter 160 is a web of thin filaments, the characteristic pore size of the filter can be determined by attempting to pass beads of different diameters through the filter and finding which diameter beads are capable of passing through the filter in large quantities. The very thin filaments can be spun onto a rotating mandrel according to U.S. Pat. No. 4,738,740 with the aid of an electrostatic field or in the absence of an electrostatic field or both. The filter thus formed can be adhered to the braid structure with an adhesive or the braid can be placed on the mandrel and the filter spun over it, or under it, or both over and under the braid to essentially capture it. The filter can have some pores formed from spraying or electrospinning and then a secondary step where pores are laser drilled or formed by a secondary operation. In one embodiment a material capable of being electrostatically deposited or spun is used to form a filter on the braid, with the preferred material being capable of bonding to itself. The filter may be made of polyurethane, pellethane, polyolefin, polyester, fluoropolymers, acrylic polymers, acrylates, polycarbonates, silicone, and/or other suitable material. The polymer is spun onto the braid in a wet state, and therefore it is desirable that the polymer be soluble in a solvent. In one embodiment, the filter is formed from polyurethane in a dimethylacetamide (DMA) and tetrahydrofuran (THF) solution. The polymer in solution is spun, with a preferred concentration of 5-10% solids for an electrostatic spin process and 15-25% solids for a wet spin process.

As another alternative method to polymer-coat the braid, the braid can be dip-coated to form a polymer filter onto the braid. The braid is mounted on a mandrel having the same outer diameter as the inner diameter of the fully expanded braid. The mandrel can be polytetrafluoroethylene (PTFE)-coated steel, in which the PTFE acts as a release surface. Alternatively, a non-coated mandrel may be used. It is important that inner diameter of the braid and the outer diameter of the mandrel not be spaced from each other when the braid is mounted on the mandrel. Thus, they preferably have a common diameter within a tolerance of ±0.065 mm. Keeping the entire inner braid in contact with the mandrel allows for the filaments to be evenly coated with the polymer, as subsequently described, so that the filter valve expands uniformly after the polymer dries. Alternately, the tubular braid can be mounted on an oversized mandrel (greater than the inner diameter of the braid), but such will result in an increase in the braid angle of the filaments, and thereby resize the filter valve and effect the expansion force thereof. In an alternate arrangement the braid may be mounted within a tubular mandrel having the same size as the outer diameter of the braid, provided with like tolerances described above. As yet another alternative, the braid can be mounted inside an undersized tubular mandrel (having an inner diameter smaller than the outer diameter of the braid), but such will result in a decrease in the braid angle of the filaments, and thereby also resize the filter valve and effect the expansion force thereof. The type of mandrel (solid or tubular), and the location of the braid thereon (external or internal), will affect localization of the polymer on the braid (providing a smooth internally coated filter valve for external mounting on a solid mandrel and providing a smooth externally coated filter valve for internally mounting within a tubular mandrel), and thereby alter areas of lubricity for the resulting filter valve.

Once the braid 140 is tightly mounted on (or within) the mandrel, the braid is dip coated into a polymer solution at a controlled steady rate. The solution is an elastomeric thermoplastic polymer dissolved in a solvent system with a boiling point ranging from 30-200° C. to produce a solution with a dynamic viscosity range of 50-10,000 cP. The rate of decent and accent is inversely dependent upon the viscosity of the solution and ranges from 1-100 mm/sec. The rate is critical to provide an even coating of the polymer on the braid, to allow wetting of all surfaces of the braid even at locations where the braid filaments are in contact with the mandrel and consequent wicking of the polymer coating into the braid particularly to the surface in contact with the mandrel, and to release air bubbles that may be trapped during the dipping process. By way of example, in one embodiment of the method for dipping into a thermoplastic urethane solution (Pellethane® dissolved in the solvents dimethylacetamide (DMA) and tetrahydrofuran (THF)), the rate is such that the dwell time of a 135 mm (6 inch) braid is 16 seconds. The rate is also preferably such that the polymer wicks down the length of the entire braid during withdrawal of the braid from the solution. The braid is dipped one time only into the solution to limit the thickness of the coating and thereby prevent restraint on the braid filaments and/or control smoothness of the polymer coating membrane. The controlled rate may be controlled by coupling the mandrel to a mechanized apparatus that dips and raises the braid on the mandrel at the steady and controlled rate into the polymer solution.

After the braid 140 is withdrawn from the polymer solution, the solvent is evaporated over a time frame relative and temperature range corresponding to the solvent boiling point, with higher temperatures and longer durations utilized for high boiling point solvents. All preferred polymer solutions use some DMA to control the uniformity of the coating thickness and may use THF to control the rate of solvent evaporation. The ratio of high boiling point solvents such as DMA to low boiling point solvents such as THF allows for control over the rate of transition from a lower viscosity high solvent content polymer solution to a high viscosity low solvent content polymer solution to a solid solvent free material, affecting the quality of the polymer membrane. In one method, the solvents are released in an oven heated to a temperature above the boiling point of DMA (165° C.) in order to rapidly release the DMA. A preferred time of heating at this temperature is 5 minutes which is sufficient to release the DMA. It is appreciated that THF has a substantially lower boiling point (66° C.) and will vaporize quickly without such substantial heating. Alternatively, the polymer-coated braid can be oven heated at a temperature below the boiling point of DMA, e.g., 80° C.-100° C., which will release the DMA from the coated braid, but at a slower rate than would occur above the boiling point of DMA. This temperature rapidly drives off the DMA while maintaining the integrity of the coated braid. A preferred time of heating at this temperature is 10 minutes which is sufficient to release the DMA. As yet another alternative, the polymer-coated braid can be allowed to dry ambient room temperature, which results in DMA release occurring at a slower rate than each of the above.

After the solvents have been released from the polymer-coated braid, the coated braid is cooled below a glass transition temperature of the polymer on the braid. Once cooled, the coated braid is released from the mandrel. If the mandrel is coated with PTFE, the braid may self-release from the mandrel or may be readily released. If the mandrel is uncoated, a release agent such as isopropyl alcohol (IPA) may be used to facilitate removal of the coated braid from the mandrel. The resulting elastomeric membrane filter formed on the braid may be elastically deformed over a range of 100-1000% elongation. In addition to Pellethane®, the membrane may be formed from, but not limited to, other polyether-based aromatic thermoplastic urethanes, polyether-based aliphatic thermoplastic urethanes (e.g., Tecoflex®), polyether block amides (e.g., Pebax®), styrene-isoprene-butadiene-styrene (SIBS), silicone, and other polymers. These polymers may be dissolved in appropriate solvents or heated to their melting point to form a fluid.

Depending on the polymer and coating technique, the coating can be fluid impermeable or porous. If porous, the coating can have a characteristic pore size between 10 μm and 500 μm, or more preferably between 15 μm and 100 μm, or even more preferably, less than 40 μm and yet more preferably between 20 μm and 40 μm.

In accord with various embodiments, the polymer coating 160 is located over strands 142 that have higher primer thickness (area 152a), lower primer thickness (area 152b), and/or no primer (area 152c). In accord with embodiments, the polymer coating is unevenly applied between the proximal and distal ends of the braided tubular form.

In yet other embodiments, the coating on the braid of filaments can be non-polymeric and applied by a suitable method. By way of example, the coating may include a metallic mesh. By way of another example, the coating may comprise a biological tissue material. Such coating materials are constructed when applied to the braid of filaments to form a barrier to passage of a therapeutic agent on the constructed occluder.

The polymer-coated braid is also preferably provided with a hydrophilic coating, hydrophobic coating, or other coating that affects how proteins within blood adhere to the filter. More specifically, the coating is resistant to adhesion of blood proteins. Suitable coatings include ANTI-FOG COATING 7-TS-13 from Hydromer, Inc. of Branchburg, NJ, and SERENE COATING from Surmodics, Inc, of Eden Prairie, MN. These and other coatings can be applied to the filter by, e.g., dipping, spraying, or roll or flow coating.

After the tubular braid is polymer coated, it is assembled to the catheter 112. The plurality of braided strands 142, the primer and the polymer filter 160 in tubular form have an inner surface and an outer surface. In one embodiment, a first end 170 of the tubular form is fixed to the catheter 112 at a first location 172 proximally adjacent the distal tip 122 of the catheter. Then the tubular form is reshaped by everting the tubular form and bending it back such that the previously inner surface of a second end 174 of the catheter now faces outward on the tubular form. The second end is then coupled to the catheter at a second location 176 proximally displaced from the first location to define a shape that flares outward in a proximal to distal direction and maximizes to a largest diameter at a central portion 178.

In an embodiment, the polymer coating is removed or perforated from portions of the occluder distal of the maximum expanded diameter of the occluder. This provides an opening into the occluder which is important for operation of the embodiment of the occluder. Such coating removal or perforation exposes the strands of the braid.

The resultant microvalve occluder is dynamically responsive to local pressure about the occluder. That is, when fluid pressure is at a determined level higher on the proximal side of the occluder than on the distal side of the occluder, the occluder automatically moves into a collapsed (or partially collapsed) configuration (closed position) with a smaller diameter permitting antegrade flow of blood about the occluder and through the vessel; and, when fluid pressure is at a determined level higher on the distal side of the occluder than on the proximal side of the occluder blood, such as occurs during therapeutic infusion through the infusion lumen and out of the distal orifice, the occluder automatically moves into an expanded configuration (open position) in with a larger diameter in which the occluder contacts the vessel wall preventing reflux (regurgitation or backward flow) of therapeutic agents. The differences between the proximal and distal pressures for dynamic operation of the microvalve occluder is based, at least in part, on the radial expansion force of the braid, as discussed above.

As indicated above, the deployable cover 124 is provided for shielding portions of the occluder 118 from the patient's vessel wall during movement of the occluder relative to the vessel wall, such as during tracking and navigation to the target location of the occluder to reduce or prevent vascular spasm prior to therapeutic delivery. The cover is preferably constructed from a polymer that is thin, strong, lubricious, and flexible. Exemplar polymers include silicone rubber, natural and synthetic rubbers, and other elastomers such as pellethane, aliphatic polyether polyurethanes, such as Tecoflex® and Corothane®, and thermoplastic vulcanizates such as Santoprene®, and styrene-isobutane-styrene (SIBS). Other materials include polyethylene terephthalate (PET), nylon, and polyimide may also be used. In addition, the cover can be constructed from a spun polymer fabric. Exemplar materials for a spun polymer fabric include pellethane and polytetrafluoroethylene (PTFE). The cover can be impermeable, semi-permeable or permeable. It is preferable to shield at least the braided strands exposed at the distal portion of the occluder as the strands are structured to cause the most friction against the vessel wall.

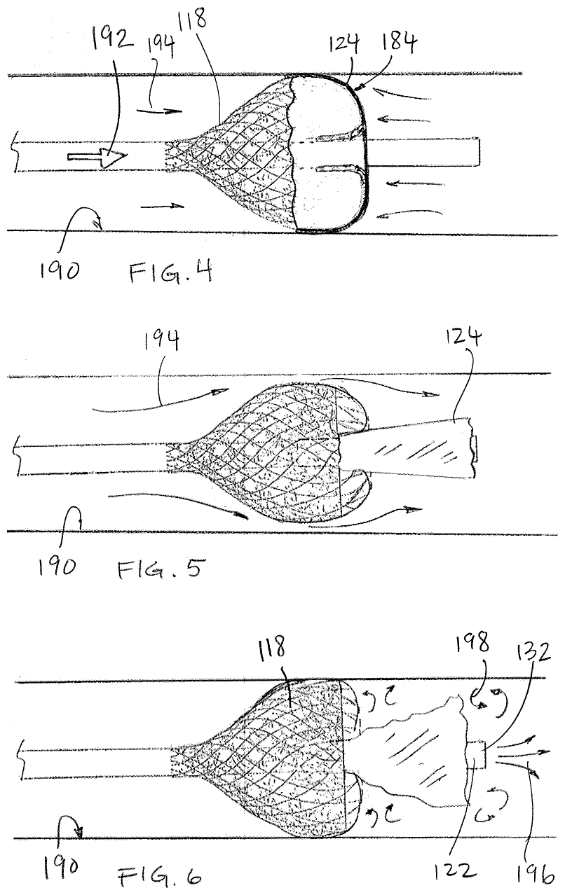

In one embodiment, a portion 180 of the cover 124 is attached to the catheter by sandwiching a portion of the cover between the outer surface 182 of the catheter 112 and the braid of the occluder 118. More particularly, the cover portion 180 is attached between the first end 170 of the occluder and the first location 172 on the catheter. The cover 124 is structured with a slight bias or memory, such that when in blood or a fluid with properties substantially the same as blood, the cover 124 is biased to expand open and generally toward the distal portion of the occluder 118. Moreover, referring to FIG. 4, when the occluder 118 is moved forward within the patient's vessel 190 in the direction of arrow 192, the fluid pressure on the distal surface 184 of the cover 124 overcomes any force on the cover from antegrade fluid flow 194 and pushes the cover into conformity with the distal portion of the occluder and particularly over the portions that would otherwise be in contact with the vessel wall. As the cover 124 has substantially greater lubricous properties than the occluder itself, friction between the occluder and the vessel wall during forward advancement of the occluder is greatly reduced. Turning to FIG. 5, then when forward motion is stopped, the fluid pressure in the vessel 190 from the antegrade flow 194 partially collapses the occluder to provide a space between the outside of the occluder and the vessel wall and overcomes the expansion force of the cover 124 to cause the cover to extend distally away from the occluder. As such, the cover does not interfere with the occluder while the occluder is stationary in the vessel. Next, referring to FIG. 6, when a therapeutic agent 196 is infused through the infusion lumen of the catheter and out of the distal orifice 132, eddy currents and turbulence 198 are generated near the distal tip 122 of the catheter. However, increased fluid pressure from the infusion causes the occluder 118 to fully expand into contact with the vessel wall 190 and prevent fluid flow past the occluder. As such, while the cover 124 may flutter in the turbulence, the cover 124 again does not interfere with operation of the occluder. Once infusion is stopped, the occluder 118 with cover 124 will again operate as shown and described with respect to FIG. 5.

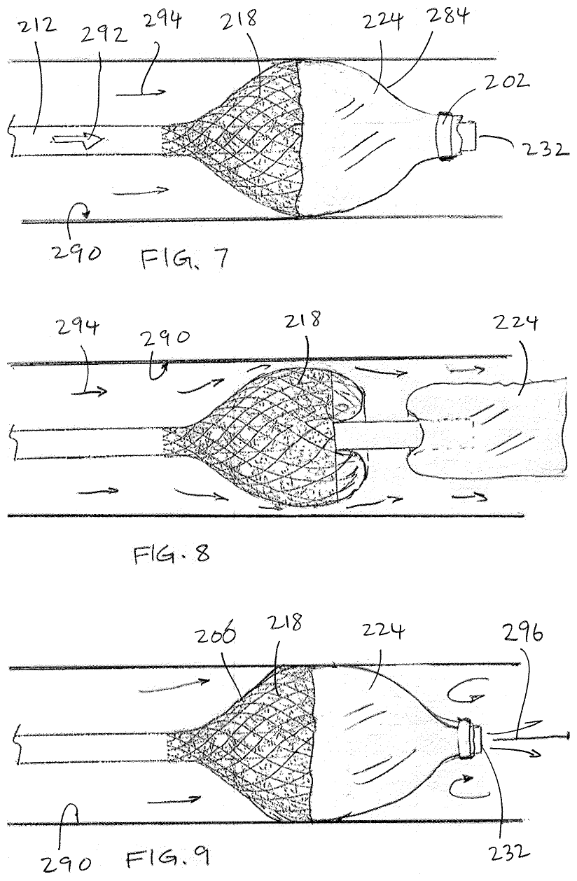

Turning to FIGS. 7 through 9, another embodiment of an infusion device 210, substantially similar to the infusion device 110 (with like parts having reference numerals incremented by 100) is shown. Infusion device 210 includes a cover 224 attached to the catheter 212 at a location displaced from the occluder 218. By way of example, the cover 224 may be attached to the catheter between occluder 218 and the distal orifice 232. The cover 224 may be attached with a crimp band 202 and/or an adhesive or bonding agent. The cover 224 is structured to extend back and toward the distal portion of the occluder. Referring to FIG. 7, when the occluder 218 is moved forward within the patient's vessel in the direction of arrow 292, the fluid pressure on the distal surface 284 of the cover overcomes any force on the cover from antegrade fluid flow 294 and keeps the cover 224 in conformity with the distal portion of the occluder 218 and particularly over the portions that would be in contact with the vessel wall 290. Turning to FIG. 8, then when forward advanced is stopped, the fluid pressure from the antegrade flow 294 moves the occluder 218 and moves the cover 224 away from the occluder 218. The cover 224 may partially collapse as well or even invert, as shown. As such, the cover 224 does not interfere with the occluder 218 and fluid flow in the vessel while the occluder is stationary in the vessel. Referring to FIG. 9, when a therapeutic agent 296 is infused through the infusion lumen of the catheter and out of the distal orifice 232, the higher pressure at the distal orifice than at the proximal side of 206 the occluder 218 cause the occluder to expand to close off fluid flow in the vessel 290 past the occluder 218 and allow the cover 224 to extend back over the distal side of the occluder. Once infusion is stopped, the occluder with cover will again operate as shown and described with respect to FIG. 8. After the therapeutic agent has been infused and the intravessel procedure finished, the infusion device 210 is removed from the patient.

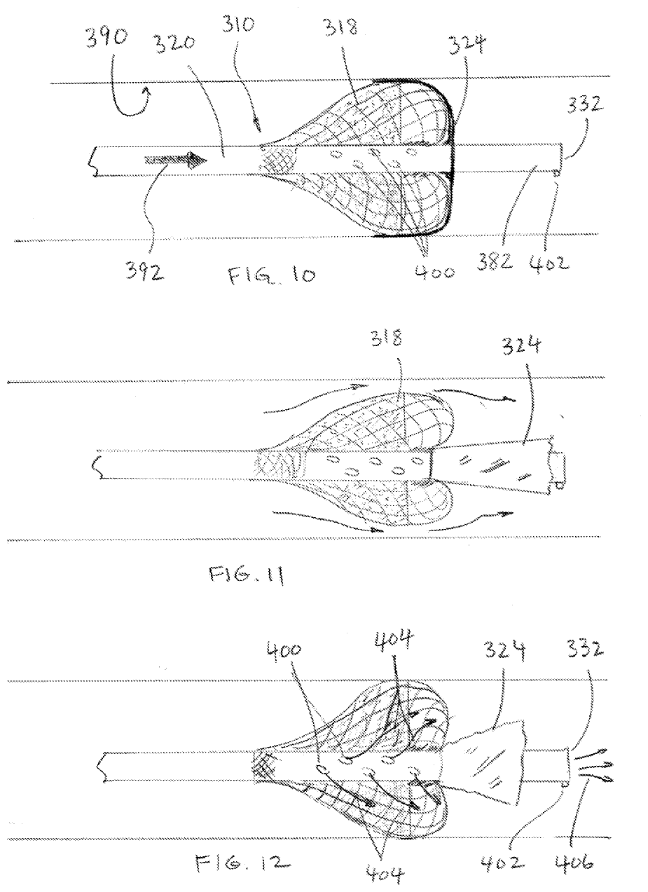

Turning now to FIGS. 10 through 12, another embodiment of an infusion device 310, substantially similar to the infusion device 110 shown in FIGS. 4 through 6 (with like parts having reference numerals incremented by 200) is shown. Infusion device 310 includes an occluder 318 mounted on a catheter 320, and provided with a cover 324 to facilitate reduced friction during advancement of the occluder through the vessel. The catheter 320 is provided with a plurality of side holes 400 extending through a wall of the catheter; the occluder 318 is provided over the portion of the catheter 320 with the side holes 400. The side holes are preferably radially oriented. The side holes are preferably evenly circumferentially displaced. By way of example and not by limitation, four to twelve catheter side holes 400 can be spaced along the portion of the catheter within the occluder. The catheter 320 additionally includes a distal orifice 332. The catheter 320 preferably includes a fluid pressure sensor 402 located at the distal end 382 of the catheter, adjacent the distal orifice 332.

The occluder 318 is advanced within the patient's vessel 390 in the direction of arrow 392, and the cover 324 operates as described above. When movement within the vessel is stopped, the cover 324 on the device further operates as described above, specifically moving away from the occluder when the pressure differential on opposing sides of the occluder 318 permits antegrade flow about the occluder, as shown in FIG. 11. Then, referring to FIG. 12, during infusion of an agent through the infusion lumen of the catheter, a portion 404 of the agent exits the holes 400 of the catheter inside the occluder 318 and generates pressure within the occluder which displaces the cover 324 from the occluder. The holes 400 are sized and numbered to have a larger surface area than the distal orifice 332; as such, there is always higher flow from infusion through the side holes 400 than the flow 406 at the distal orifice 332, which prevents the cover 324 from the blocking the occluder 318.

Further, with end hole infusion devices it is common to have significant turbulence and venturi effects near the distal orifice. Such turbulence can cause localized pressure drops and result in errors in pressure readings in the vessel. However, the provision of the side holes 400 and location thereof, i.e. away from the pressure sensor and on an opposite side of the cover 324 from the sensor 402 as shown in FIG. 12, permits the pressure sensor 402 to obtain pressure readings that are more stable and less prone to the artifacts of localized turbulence. This advantage is provided even if the cover were eliminated from the embodiment, and this disclosure contemplates that an embodiment may be implemented with or without the cover to obtain advantages of the side holes including, but not limited to, reducing turbulence at the distal orifice and reducing the artifacts of the turbulence on readings from a pressure sensor at a distal end of the catheter.

While the above description has been primarily directed to use of the device for infusing a therapeutic agent, it is appreciated that the device has functionality even when delivery of a therapeutic agent is not the primary function.

There have been described and illustrated herein embodiments of devices and methods for reducing vessel spasm while infusing a therapeutic agent with a infusion device with an occluder or performing other procedures in a vessel. While particular embodiments of the invention have been described, it is not intended that the invention be limited thereto, as it is intended that the invention be as broad in scope as the art will allow and that the specification be read likewise. Thus, while various materials for the cover, microvalve filaments, the valve coating, and the catheter have been described, it will be appreciated that other materials can be utilized for each of them in each of the various embodiments in combination and without limitation. While the occluder is described as a preferred microvalve, the cover can also be used with other occluders including static occluders, including balloons and malecots. Also, while infusion of a therapeutic agent has been referred to herein throughout, a therapeutic agent should be considered broadly including any treatment agent, including, not by limitation, drugs that target cancer cells and immunotherapy agents, including immunomodulators, vaccines, modified cells and check-point inhibitors, as well as agents aiding in the delivery of treatment agents, including but not limited to contrast agents. Also, while the invention may have been described with respect to particular vessels of humans, it will be appreciated that the invention can have application to any blood vessel and other vessels, including ducts, of humans and animals. In particular, the apparatus can also be used in treatments of tumors, such as liver, renal or pancreatic carcinomas. Further, the embodiments have been described with respect to their distal ends because their proximal ends can take any of various forms, including forms well known in the art. It will therefore be appreciated by those skilled in the art that yet other modifications could be made to the provided invention without deviating from its scope as claimed.

What is claimed is:

1. A therapeutic device for delivery of a therapeutic agent within a vessel of a patient, the vessel having a vessel wall, the device comprising:
a) a catheter having a proximal end and distal end;
b) a vessel occluder mounted adjacent the distal end of the catheter; and
c) a flexible cover attached to the catheter and adapted to extend between a portion of the occluder and the vessel wall when the occluder is advanced through the vessel, and automatically move away from the occluder when predetermined pressure conditions inside the vessel are met.

2. The therapeutic device according to claim 1, wherein a portion of the cover is sandwiched between the outer surface of the catheter and a distal end of the occluder.

3. The therapeutic device according to claim 1, wherein the cover is fixed to the catheter at a location distally of a distal end of the occluder.

4. The therapeutic device according to claim 1, wherein the cover is self-expanding.

5. The therapeutic device according to claim 1, wherein the cover is made from lubricous material.

6. The therapeutic device according to claim 1, wherein the cover is impermeable.

7. The therapeutic device according to claim 1, wherein the cover is semi-permeable.

8. The therapeutic device according to claim 1, wherein the cover is made from an elastomeric material.

9. The therapeutic device according to claim 1, wherein the cover is made from a polymeric material.

10. The therapeutic device according to claim 9, wherein the polymer is at least one of pellethane and polytetrafluoroethylene.

11. The therapeutic device according to claim 1, wherein the catheter a distal orifice and side holes proximal of the distal orifice, and the occluder extends over the side holes.

12. The therapeutic device according to claim 11, further comprising a pressure sensor at the distal end of the catheter.

13. The therapeutic device according to claim 12, wherein the cover is located between the side holes and the pressure sensor.

14. A therapeutic device for delivery of a therapeutic agent within a vessel of a patient during an vascular procedure, the vessel having a vessel wall, comprising:
a) a flexible catheter sized for introduction into the vessel, the catheter having a proximal end and a distal end, an outer surface, a lumen extending between the proximal and distal ends and opening at a distal orifice; and
b) an occluder mounted adjacent the distal end of the catheter, the occluder having a proximal side and a distal side, the occluder adapted to dynamically change shape in response to the relative fluid pressure at the proximal and distal sides of the occluder, wherein,
at a first fluid pressure condition in the vessel with fluid pressure higher on the proximal side than on the distal side of the occluder, the occluder moves into a first configuration with a first diameter permitting proximal to distal flow of blood about the occluder and through the vessel, and,
at a second fluid pressure condition in the vessel with fluid pressure higher on the distal side than on the proximal side of the occluder, the occluder moves into a second configuration with a relatively larger second diameter sized to contact the vessel wall and preventing the passage of fluid in the vessel and past the occluder; and
c) a flexible cover attached to the catheter and adapted to extend between a portion of the occluder and the vessel wall when the occluder is advanced through the vessel, and move away from the occluder when the second fluid pressure conditions are present.

15. The therapeutic device according to claim 14, wherein the occluder has,
i) a plurality of elastic filamentary strands in a tubular braid, the strands having proximal portions, central portions, and distal portions,
the distal portions longitudinally fixed to the outer surface of the catheter at the first location,
the proximal portion longitudinally fixed to the outer surface of the catheter at the second location, and
the central portions biased to extend radially outward from the outer surface between the proximal and distal portions; and
ii) a polymeric material coated onto the filamentary strands to form a barrier to passage of the therapeutic agent.

16. The therapeutic device according to claim 15, wherein:
the polymeric material is coated onto at least proximal portions of the strands and at least a portion of the central portions of the strands, and the distal portions of the strands sufficiently absent of the polymeric material such that the therapeutic agent can flow through the occluder between the distal portions.

17. The therapeutic device according to claim 15, wherein the distal portions of the strands are inverted to be at least partially located radially within the central portions of the strands.

18. The therapeutic device according to claim 15, wherein the strands are made of nickel titanium alloy.

19. A therapeutic device for delivery of a therapeutic agent within a vessel of a patient during a vascular procedure, the vessel having a vessel wall, comprising:
a) a flexible catheter sized for introduction into the vessel, the catheter having a proximal end and a distal end, an outer surface, a lumen extending between the proximal and distal ends and opening at a distal orifice and at a plurality of side holes proximal of the distal orifice; and
b) an occluder mounted adjacent the distal end of the catheter, the occluder having a proximal side and a distal side, the occluder extending over the side holes, the occluder adapted to dynamically change shape in response to the relative fluid pressure at the proximal and distal sides of the occluder, wherein,
at a first fluid pressure condition in the vessel with fluid pressure higher on the proximal side than on the distal side of the occluder, the occluder moves into a first configuration with a first diameter permitting proximal to distal flow of blood about the occluder and through the vessel, and,
at a second fluid pressure condition in the vessel with fluid pressure higher on the distal side than on the proximal side of the occluder, the occluder moves into a second configuration with a relatively larger second diameter sized to contact the vessel wall and preventing the passage of fluid in the vessel and past the occluder.

20. The therapeutic device according to claim 19, further comprising a pressure sensor at the distal end of the catheter.

21. A therapeutic device for delivery of a therapeutic agent within a vessel of a patient during a vascular procedure, the vessel having a vessel wall, comprising:
a) a flexible catheter sized for introduction into the vessel, the catheter having a proximal end and a distal end, an outer surface, a lumen extending between the proximal and distal ends and opening at a distal orifice and at a plurality of side holes proximal of the distal orifice; and
b) an occluder mounted adjacent the distal end of the catheter, the occluder having a maximum diameter and a proximal fluid impermeable side and a distal fluid permeable side located on opposite sides of the maximum diameter, the occluder extending over the side holes, the occluder adapted to dynamically change shape in response to the relative fluid pressure at the proximal and distal sides of the occluder.

22. The therapeutic device according to claim 21, further comprising a pressure sensor at the distal end of the catheter.

23. The therapeutic device according to claim 22, wherein the pressure sensor is located distal of the occluder.
